(19) United States
(12) Patent Application Publication
(10) Pub. No.: US 2009/0287087 A1
(43) Pub. Date: Nov. 19, 2009

(54) DEVICES FOR CREATING PASSAGES AND SENSING FOR BLOOD VESSELS

(75) Inventors: Eric J. GWERDER, Fremont, CA (US); Thomas M. KEAST, Sunnyvale, CA (US); Edmund J. ROSCHAK, Mission Viejo, CA (US)

Correspondence Address: Broncus Technologies, Inc. c/o;Levine Bagade Han LLP, 2400 Geng Road, Suite 120, Palo Alto, CA, 94303, US

(73) Assignee: Broncus Technologies, Inc., Mountain View, CA (US)

(21) Appl. No.: 12/469,614

(22) Filed: May 20, 2009

Related U.S. Application Data
(63) Continuation of application No. PCT/US2007/084330, filed on Nov. 9, 2007.
(60) Provisional application No. 60/867,076, filed on Nov. 22, 2006.

(51) Int. Cl. A61B 8/14; A61M 29/00

(52) U.S. Cl. 600/463; 606/191

(57) ABSTRACT

Devices and methods are disclosed for creating passages in tissue and detecting blood vessels in and around the passages. The devices may be used to create opening in tissue without removing a sensing assembly from the tissue. The devices herein may be used for altering gaseous flow within a lung to improve the expiration cycle of an individual, particularly individuals having Chronic Obstructive Pulmonary Disease (COPD). In addition, the devices may be used to sample tissue during biopsy or other medical procedures where perforating a blood vessel could result in injury to a patient.

### CROSS-REFERENCE TO RELATED APPLICATIONS

This application is a continuation of International Application No. PCT/US2007/084330, filed Nov. 9, 2007 which claims priority to U.S. Provisional Application No. 60/867,076, filed Nov. 22, 2006; both applications are incorporated herein by reference in their entireties.

### FIELD OF THE INVENTION

The invention is directed to devices for sensing movement within tissue, and then creating passages in the tissue. In one variation, sensing movement may comprise sensing for the presence or absence of blood vessels. Variations may include further detecting for these blood vessels in the passages created by the device.

### BACKGROUND OF THE INVENTION

It was found that creation of collateral channels in COPD patients allowed expired air to pass out of the lungs and decompressed hyper-inflated lungs. Such methods and devices for creating and maintaining collateral channels are discussed in U.S. Pat. No. 6,692,494; U.S. patent application Ser. Nos. 09/947,144, 09/946,706, and 09/947,126 all filed on Sep. 4, 2001; US patent application No. filed on Sep. 4, 2002; U.S. patent application Ser. No. 11/335,263, filed on Jan. 18, 2006; Attorney Docket number BRON-N-E004.05-US, U.S. patent application Ser. No. ______ and filed on Nov. 22, 2006: each of which is incorporated by reference herein in its entirety.

The creation of these channels also seems to overcome the shortcomings associated with bronchodilator drugs and lung volume reduction surgery. Placement of an implant within the channel further increased the duration of the treatment.

However, because creation of the opening/channel is typically performed within the airway under bronchoscopic observation, care must be taken so as not to rupture a pulmonary vessel that lies beneath or outside of the airway wall. The need to avoid rupturing vessels that may be hidden by the airway walls is also evident when a surgeon attempts to obtain a biopsy sample from within the bronchial tree. In addition, because the location of the pulmonary vessels varies between patients, care must also be taken when working within the channel or biopsy site. For instance, although a channel may be created without puncturing a blood vessel, the subsequent dilation, insertion of an implant, and/or removal of biopsy material may perforate vessels that were otherwise undetected during the creation of the channel.

The problem is compounded when accounting for the tidal motion of lungs. For example, because the target site moves due to the tidal motion of the lungs (as a result of the mechanics of breathing), it is difficult to visually identify an area that was previously scanned unless the scanning device remains relatively stationary against the tissue. Moreover, the difficulty increases when considering that the procedure takes place through the camera of a bronchoscope or endoscope.

In view of the above, a need remains to increase the safety when creating openings in tissue so as not to rupture a blood vessel. Such a device may have applications outside of the lung in any situation where there is a need to locate blood vessels or other fluid carrying vessels prior or during creation of an opening in the tissue.

### SUMMARY OF THE INVENTION

The invention relates to creation of passages and/or removal of tissue while allowing sensing of blood vessels that may be in or around the area of the passage. Although specific reference is made to use of the subject invention within the lungs, it is noted that the invention may also be used within various other parts of the body that have a need for such safety measures.

The device allows for sensing blood vessels in tissue, and allows for creation of a passage or opening without moving the sensing element. The device includes a catheter member having a near end, a far end, and a lumen extending therethrough, a dilation member within the lumen and having a shaft, the shaft having a distal tip and a non-distensible expandable member adjacent to the distal tip, the dilation member being slidably located within the catheter member, a sensing assembly located at the far end of the catheter member, and where the non-distensible expandable member is moveably located within the lumen independent of the sensing assembly, such that when the sensing assembly contacts tissue, the dilation member may be advanced out of the catheter member and into the tissue without removing the sensing member from the tissue.

The catheter member can be a tubular member as commonly used in medical device applications. Accordingly, the catheter member can be a polymeric tube or a reinforced polymeric tube. As described herein, it may have one or lumens to accommodate the variations of the devices within this disclosure.

The dilation member is typically used to dilate the opening created by the device. As such it may be a distensible balloon or a non-distensible balloon. The advantages of each are discussed below. Variations of the device include mechanical expandable members such as baskets or other such members.

The sensing assembly is used to scan the tissue to minimize causing undesirable injury to the patient. As discussed below, any number of sensing modes may be incorporated into the device. However, it was found that Doppler ultrasound transducer assemblies perform acceptably when sensing for blood vessels within tissue. In certain variations, the sensing assembly may be configured to puncture the tissue and create the opening. However, in other variations, the sensing assembly will have a blunted tip to minimize undesirable tissue damage.

In variations of the device, the sensing assembly is offset from an axis of the catheter and/or dilation assembly. Doing so improves the ability of the sensing assembly to contact tissue surfaces when the device is advanced along body conduits. In addition, this offset feature improves the ability to see the tip of the sensing assembly when the device is used with a scope type device.

The invention further includes methods of treating tissue, where the method includes selecting an area in the tissue for treatment, advancing a device into the lung to a tissue site, where the device includes a sensing assembly affixed to a catheter and a dilation assembly located within the catheter, scanning the tissue site with the sensing assembly for the presence or absence of blood vessels, creating an opening with the device without removing the sensing assembly from the tissue site, and dilating the opening with the dilation assembly.

The methods may include treating tissue to assist in decompressing hyper inflated lung tissue. Alternatively, the methods may include scanning of tissue during a biopsy or other medical procedures where perforating a blood vessel could result in injury to a patient.

The selecting step may be performed with direct visual imaging from the scope type device and/or may be performed with various types of non-invasive imaging equipment such as: x-ray, acoustic imaging, MRI, PET, computed tomography (CT) scans or other such imaging.

The step of creating the opening with the device may include using the dilation assembly or the sensing assembly to create an opening at the treatment site or adjacent to the treatment site (but within an acceptable range so that the scanning covers the tissue being penetrated).

In certain variations, the sensing assembly may also be inserted into the opening (prior to or after dilation) to ensure that a blood vessel or other organ was not missed when scanning the surface of the tissue.

As noted herein, one variation of the device permits scanning the tissue site by placing the sensing assembly in contact with the tissue site. However, various sensing assemblies may permit non-contact scanning. Regardless of whether the sensing tip contacts the tissue, creation of the opening may be performed without significant movement of the scanning assembly. Such a benefit is apparent as medical practitioners may lose track of the scanned tissue if they are required to substitute or move the scanning assembly to create an opening.

### BRIEF DESCRIPTION OF THE DRAWINGS

FIGS. 1A-1C illustrate various states of the natural airways and the blood-gas interface.

FIG. 1D illustrates a schematic of a lung demonstrating a principle of the effect of collateral channels placed therein.

FIG. 2A shows a variation of a system as described herein.

FIG. 2B shows a far end of a catheter member with a dilation member in an actuated state.

FIGS. 3A-3F show variations of the ends of devices.

FIGS. 4A-4C show various cross sectional views of devices and the expandable member being attached to a dilation member.

FIG. 4D illustrates a variation of a catheter member having dual lumens of varying sizes to accommodate the distal end of the dilation member.

FIGS. 5A-5B illustrate a non-exhaustive sample of variations of transducer assemblies.

FIGS. 6A-6D, illustrate possible variations of optional tips for use with the transducer assembly.

FIGS. 7A-7D illustrates examples of using the device to scan, create and dilate an opening in tissue.

FIG. 7E illustrates an optional step of scanning the opening before or after dilation of the opening.

### DETAILED DESCRIPTION OF THE INVENTION

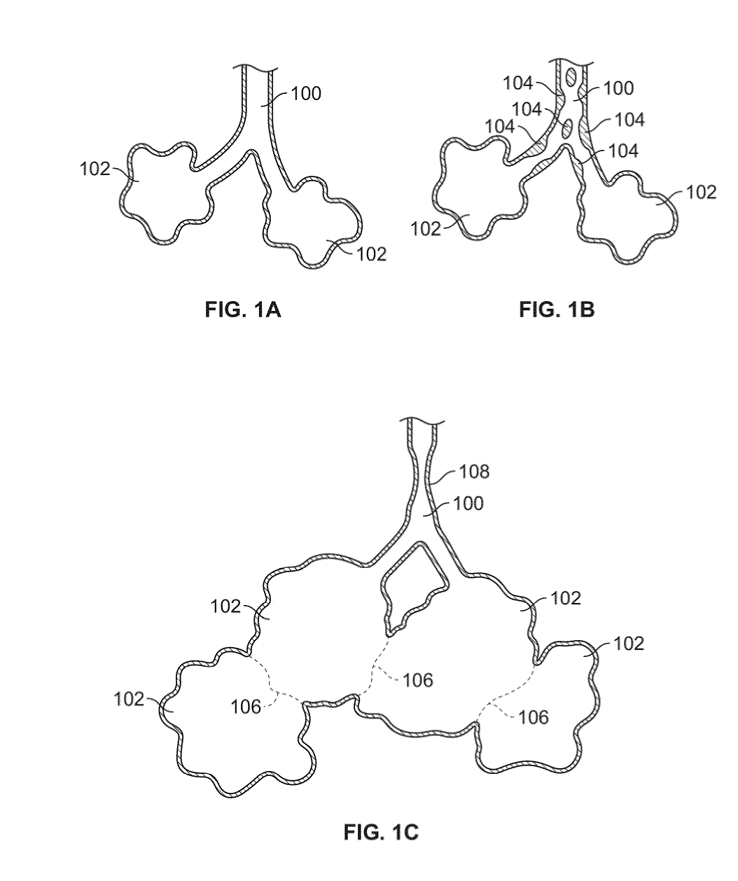

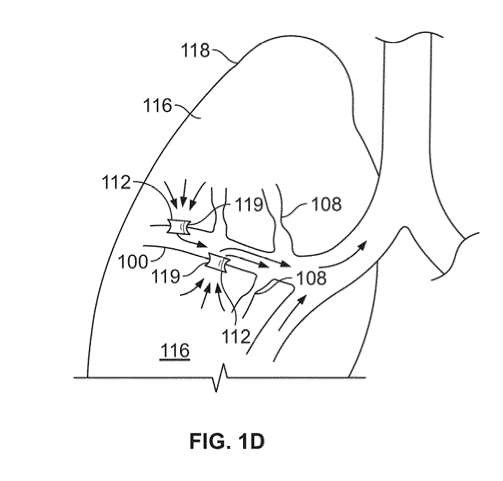

FIG. 1A shows a simplified illustration of a natural airway 100 which eventually branches to a blood gas interface 102. FIG. 1B illustrates an airway 100 and blood gas interface 102 in an individual having COPD. The obstructions 104 (e.g., excessive mucus resulting from COPD, see above) impair the passage of gas between the airways 100 and the interface 102. FIG. 1C illustrates a portion of an emphysematous lung where the blood gas interface 102 expands due to the loss of the interface walls 106 which have deteriorated due to a bio-chemical breakdown of the walls 106. Also depicted is a constriction 108 of the airway 100. It is generally understood that there is usually a combination of the phenomena depicted in FIGS. 1A-1C. More usually, the states of the lung depicted in FIGS. 1B and 1C are often found in the same lung.

As will be explained in greater detail below, the production and maintenance of collateral openings or channels through airway walls permits expired air to pass directly out of the lung tissue and into the airways to ultimately facilitate exchange of oxygen into the blood and/or decompress hyper inflated lungs. The term ‘lung tissue’ is intended to include the tissue involved with gas exchange, including but not limited to, gas exchange membranes, alveolar walls, parenchyma, airway walls and/or other such tissue. To accomplish the exchange of oxygen, the collateral channels allow fluid communication between an airway and lung tissue. Therefore, gaseous flow is improved within the lung by altering or redirecting the gaseous flow within the lung, or entirely within the lung.

FIG. 1D illustrates a schematic of a lung 118 to demonstrate a benefit of the production and maintenance of collateral openings or channels through airway walls. As shown, a collateral channel 112 (located in an airway wall 110) places lung tissue 116 in fluid communication with airways 100 allowing expired air to directly pass out of the airways 100. The term channel is intended to include an opening, cut, slit, tear, puncture, or any other conceivable artificially created opening. As shown, constricted airways 108 may ordinarily prevent air from exiting the lung tissue 116. In the example illustrated in FIG. 1D, there is no implanted structure placed in the collateral channel 112. However, conduits or implants 119 may be placed in the collateral channels 112 to assist in maintaining the patency of the collateral channels 112. Examples of conduits may be found in the applications discussed above. While there is no limit to the number of collateral channels which may be created, it is preferable that 1 or 2 channels are placed per lobe of the lung. For example, the preferred number of channels is 2-12 channels per individual patient. In current trials, it was found that 1-4 channels placed per lobe of the lung and 4-16 channels per individual patient was preferable. This number may vary on a case by case basis. For instance, in some cases an emphysematous lung may require 3 or more collateral channels in one or more lobes of the lung.

The present invention includes a device which is able to detect the presence or absence of a blood vessel by placing a front portion of the device in contact with tissue and then create an opening in the tissue without having to remove the device from the tissue.

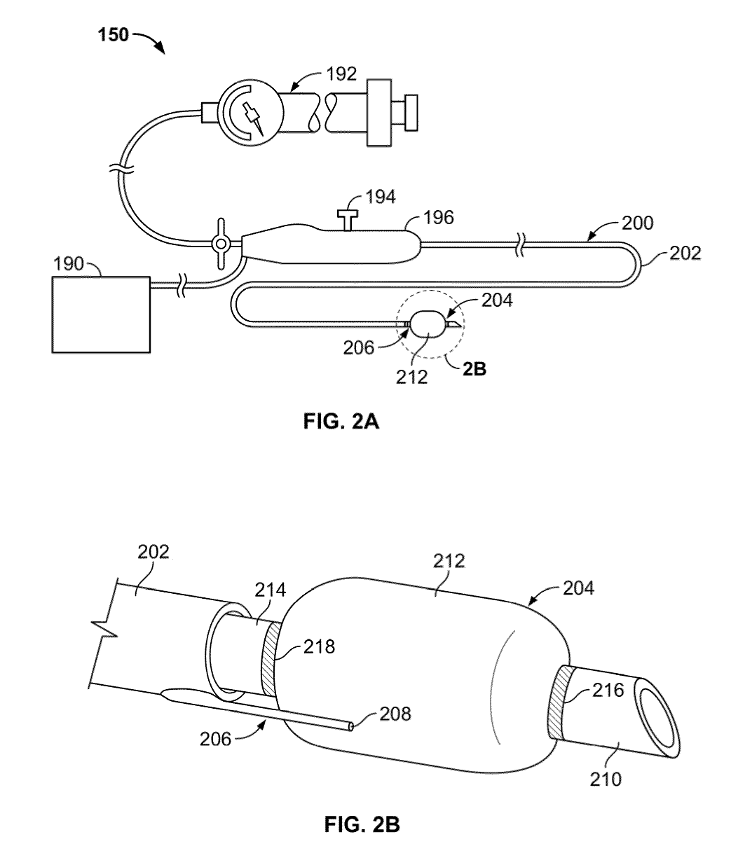

FIG. 2A illustrates a sectional side view of a variation of the inventive system 150. The system generally includes a device 200 for sensing the presence of blood vessels and creating passages in tissue. The device 200 includes a catheter member 202 and dilation member 204 extending through the catheter member 202. In the variation shown, the dilation member 204 is shown in an actuated position outside of the catheter member 202. However, as discussed herein, the dilation member 204 is moveable relative to the catheter member 202 and sensing assembly 206 so that it can be withdrawn relative to the sensing assembly 206 and/or the catheter member 204. In this variation, the dilation member 204 is coupled to an actuator 194 that is slidably located on a handle 196 at a near end of the catheter 202. However, in alternate variations, the dilation member may also extend from the near end of the catheter member 202. In such a case the medical practitioner moves the dilation member 204 relative to the catheter member 202 to insert the dilation member 204 into the tissue.

Typically, the catheter member 202 is sufficiently flexible and has a length that allows for the far end of the catheter member 202 to reach target sites when the device 150 enters the body through a bronchoscope or endoscope. Some variations of devices described herein can be constructed to be stiff and inflexible. However, for most procedures, the device has sufficient flexibility, column strength and length to access the tissue targeted for treatment within tortuous anatomy (such as those devices intended for use in small airways of the lung). Accordingly, for devices used to create collateral channels within lungs, the length of the device should preferably be between 1.5-5 ft long (preferably 4-5 ft) in order to reach the targeted airways.

The device 200 may be coupled to a control system 190 that is configured to assist the medical practitioner in detecting whether blood vessels are at or near a particular target site. The system 150 also includes a fluid source 192 for dilation of the tissue after the device creates the openings. The fluid source may be any standard device used to pressurize gas or liquid into an expandable dilation member 212 located at the far end of the device 200. Although illustrated to be a syringe type device, the fluid source 192 may a compressor type device as well.

When used, the control system 190 is coupled to a sensing assembly 206 that extends from the far end of the catheter member 202. The sensing assembly 206 and control system 190 may be any type of unit that confirms the presence or absence of blood vessels. As such, it may be a thermal based system, light based system, ultrasound based system, or Doppler based system. For exemplary purposes, the control system 190 and sensing assembly 206 are discussed herein as being a Doppler ultrasound system. As such, the sensing assembly 206 includes a sensing tip 208 that is coupled to the power supply 190 as is known by those familiar with such systems. For example, the sensing assembly 206 may include any number of conducting members (e.g., wires) extending along the catheter member 202 (either internally or externally to the catheter member 202). In any case, these conducting members provide the energy and controls for the sensing assembly 206. In the case of Doppler ultrasound, the conducting members couple an ultrasound source 190 to the sensing tip 208 that comprises an ultrasound transducer assembly or lens.

Moreover, variations of the inventive device include conducting members that comprise a series of wires, with one set of wires being coupled to respective poles of the transducer, and any number of additional sets of wires extending through the device. In addition, the sensing assembly 206 may have more than one sensing surface disposed along the portion of the sensing assembly 206 that extend from the device.

As discussed herein, any conventional sensing type probe may be used to detect the blood vessel. When using Doppler ultrasound to detect the presence of blood vessels within tissue, the ultrasound can operate at any frequency in the ultrasound range but preferably between 2 Mhz-30 Mhz. It is generally known that higher frequencies provide better resolution while lower frequencies offer better penetration of tissue. In the present invention, because location of blood vessels does not require actual imaging, there may be a balance obtained between the need for resolution and for penetration of tissue. Accordingly, an intermediate frequency may be used (e.g., around 8 Mhz). A variation of the invention may include inserting a fluid or gel into the airway to provide a medium for the Doppler sensors to couple to the tissue to detect blood vessels. In those cases where fluid is not inserted, the device may use mucus found within the airway to directly couple the sensor to the wall of the airway.

As noted above, Doppler ultrasound was found to be an efficient way to identify blood vessels. As such, the control system 190 can be configured to communicate with an analyzing device or control unit adapted to recognize the reflected signal or measure the Doppler shift between the signals. The source signal may be reflected by changes in density between tissues. In such a case, the reflected signal will have the same frequency as the transmitted signal. When the source signal is reflected from blood moving within a vessel, the reflected signal has a different frequency than that of the source signal. This Doppler Effect permits determination of the presence or absence of a blood vessel within tissue. The Doppler system described herein comprises a Doppler ultrasound mode of detection. However, additional variations include transducer assemblies that allows for the observation of the Doppler Effect via light or sound as well.

Regardless of the mode incorporated by the sensing assembly the system 150 may include a user interface that allows the user to determine the presence or absence of a blood vessel at the target site. Typically, the user interface provides an audible confirmation signal. However, the confirmation signal may be manifested in a variety of ways (e.g., light, graphically via a monitor/computer, etc.)

Although depicted as being external to the device, it is contemplated that the control system 190 may alternatively be incorporated into the device 200. Moreover, the system 150 may incorporate any number of connectors or fitting that allow for either permanent or detachable connections of the fluid source, control system and/or any other auxiliary systems used with the system 150.

FIG. 2B illustrates an expanded view of the dilation member 204 extending out of the far end of the catheter member 202. As shown, the dilation member includes a distal tip 210 and an expandable member 212 located adjacent to the tip 210. In the illustration shown, the tip 210 comprises a sharpened tip. When the tip 210 comprises such a tissue piercing configuration, the tip 210 is able to penetrate soft tissue or other composite type tissue (e.g., that of an airway wall). Such a configuration may include a stainless steel thin walled tubing such as a hypo-tube, cannula tubing such as that used for needles, solid sharpened mandrel, double beveled needle tip, etc. located at a far end of a shaft 214 of the dilation member 204. The tip 210 described herein will be sharp or have a sufficiently small surface area such that insertion of the tip 210 through tissue occurs by advancement of the dilation member 210 (or a component thereof). It is contemplated that, where possible, any of the tissue piercing tips described herein may be incorporated into any of the variations described herein. In alternate variations, the tip 210 may be rounded or blunted. Regardless, the tip 210 is configured to facilitate entry of the expandable member 212 into tissue for dilation of the tissue (whether the opening is created by the tip 210 or the sensing assembly 206).

FIG. 2B also illustrates the dilation member 204 having an expandable member 212. Although the expandable member 212 is shown as being a balloon, alternate expandable members (such as those that allow for mechanical dilation such as a basket, a dilator, etc.) are within the scope of the disclosure. In such a case, the fluid source 192 is naturally replaced with the appropriate actuation mechanism. The use of a balloon 212 allows for controlling pressure during dilation of the passage in tissue created by the tip 210.

Variations of the device 200 can be designed for use in tough tissue that is resistant to radial expansion (such as an airway wall). In such variations, the balloon may comprise non-distensible balloon to overcome the toughness of the tissue. Non-distensible balloons are generally made up of relatively inelastic materials consisting of PET, nylons, polyurethanes, polyolefins, PVC, and other crosslinked polymers. Therefore, use of a non-distensible balloon allows for easier expansion of tissue because the non-distensible balloon permits high pressurization (>6 atm). Moreover, non-distensible balloons generally inflate in a uniform shape (radially longitudinally, or both) since the balloon unfolds to assume an expanded shape. In contrast, distensible balloons typically expand in shape when pressurized. In any case it should be noted that distensible and/or non-distensible balloons may be used in the present invention depending upon the application.

Non-distensible balloons typically occupy a greater mass than distensible balloons because the non-distensible balloon is inelastic and is folded in an unexpanded shape. Therefore, variations of the invention include non-distensible balloons having a working diameter (or diameter in an unexpanded shape) that is close to the diameter of the tip 210 or shaft 214. This allows insertion of the unexpanded balloon into the opening created by the piercing member. Accordingly, balloons of the present invention may include thin walled balloons, balloons with small distal profiles, balloons with distal ends that are close in actual diameter to the diameter of the piercing member, or balloons that folds into low profile state, or balloons having a combination of these features.

FIG. 2B also illustrates markers 216, 218 placed on the dilation member 204. In this variation, markers 216, 218 are placed on the distal and proximal ends of the expandable member 204. However, variations include use of a single marker. The markers 216, 218 may be radiopaque, and/or visually apparent. In the latter case, a visually apparent marker permits the medical practitioner to confirm location of the expandable member 212 prior to dilation. The marker 218 located at the proximal end of the expandable member 212 assists the practitioner in determining when the expandable member 212 clears the catheter member 202.

The markers 216, 218 may be a ring of biocompatible polymer and may be selected to provide contrast so that it may be identified as the medical practitioner views the device through an endoscope or bronchoscope. For example, the bronchoscope will usually contain a light-source that illuminates the target area. Therefore, the markers may be fabricated to reflect or refract the light in a different manner from the remainder of the device. In one variation, the markers may be the same color as the remainder of the device, or partially transparent, or entirely transparent, but is identifiable because the markers reflect or refracts light differently than the remainder of the device.

The markers may be made using a number of techniques. In one example, the mark is a ring formed of silicone and is white. The polymeric ring may be spun onto the dilation member. In another example the marker is a ring formed of silicone and is black. In another example the mark is a ring formed by suspending gold particulates in a polymer allowing for visual and radiopaque contrast.

The shape of the marker is not limited to a thin ring. The visualization mark may be large. The markers may, for example, be a white coating disposed on the shaft of the dilation member. It should be noted that variations of the invention include coloring the balloon itself, or other expandable member, to provide contrast like the marker.

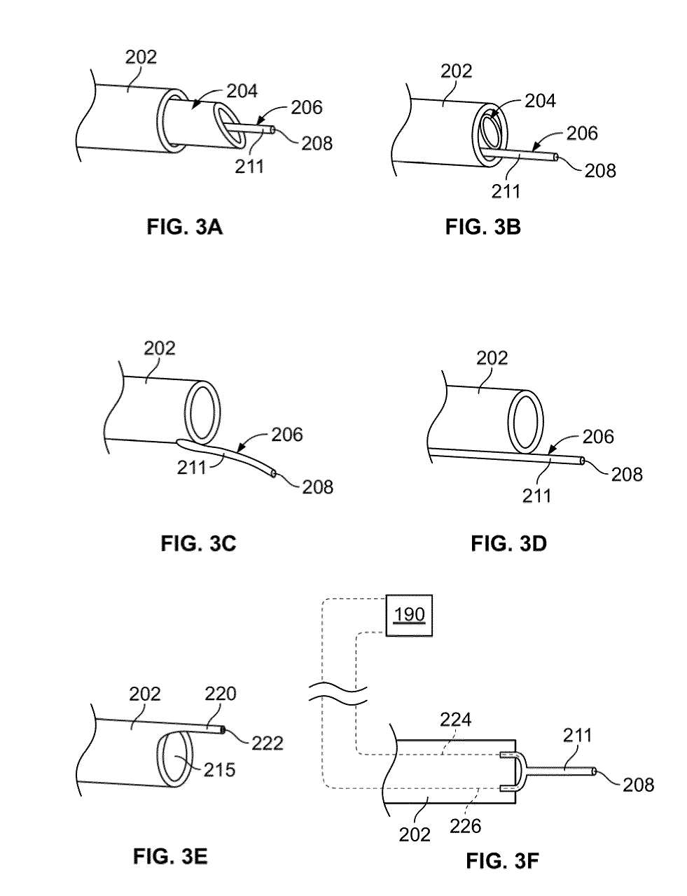

FIGS. 3A to 3B illustrate various configurations of the far end of the catheter member 202 to illustrate variations of sensing assembly 206 configurations.

FIG. 3A shows a variation of a sensing assembly 206 having a segment 211 that extends from within the tip of the dilation member 204. As discussed herein, in most variations, the sensing tip 208 is fixed and extends a distance beyond the catheter 202 so that the tip 208 may be pressed against tissue to scan for blood vessels or other structures. Once the practitioner locates an acceptable site, the practitioner advances the dilation member to create an opening with the distal tip 210. Although placing the sensing assembly 208 through the dilation member 204 may offer more precise scanning of the tissue prior to creation of any openings, this construction may be more complicated as the added structure of the sensing assembly 204 may make it more difficult to navigate the catheter member 202 through tortuous anatomy.

FIGS. 3B-3F illustrate variations of sensing assemblies 206 in which the segments 211 are offset from a central axis of the catheter member 202. It is noted that the variation of FIG. 3A may also comprise an offset segment 211 construction while remaining within the distal tip of the dilation member. In any case, the offset feature is useful when navigating the device 200 through tortuous anatomy. In one aspect, when the offset feature is outside of the dilation member, it eliminates the need for the segment 211 from extending through the length of the catheter member. All constructions of the offset feature also reduces the chance that the sensing tip 208 will be obscured by the catheter member 202 when viewed by the end of the bronchoscope or endoscope.

FIG. 3B illustrates another variation of a sensing assembly 206 extending from the far end of a catheter member 202. In this variation, the segment 211 of the sensing assembly 206 is affixed within the catheter member 202 but externally to the dilation member 204. The segment 211 may extend through the length of the catheter member 202 or may be terminated near the far end of the catheter member 202 with the conductive elements (e.g., wires) extending to the control system (not shown). In some variations, the segment 211 extends through the device but the portion extending from the far end of the catheter is stiff/has a sufficient column strength to probe tissue while a remainder of the segment has a lower stiffness/column strength to accommodate flexibility of the device. In any such constructions, the conductive element (or segment portion that extends in the device) does not significantly reduce the ability to navigate the device through tortuous anatomy.

FIG. 3C illustrates another variation of a segment 211 of the sensing assembly 206 in which the sensing tip 208 is angled away from a central axis of the catheter member 204. Such a feature is useful when trying to sense along a wall of a body passage because less articulation of the catheter 202 is required to contact the sensing tip 208 against tissue.

FIG. 3D shows another variation of an offset sensing assembly 206. In this variation, the segment 211 may comprise a tube or similar member that extends along and externally to the length of the catheter member 202.

FIG. 3E illustrates yet another variation of an offset sensing tip. However, in this variation, the catheter member 202 can include an integral projection 220 that extends distally from a first lumen 215. The projection 220 includes a second lumen 222 through which a sensing assembly (not shown) may be secured.

FIG. 3F shows another variation in which a segment 211 of the sensing assembly is inserted into the far end of the catheter member. The segment 211 may have connections for coupling to a control system as described above. In such a variation, the catheter member 202 may be a multi-lumen tube with one or more lumens reserved for the sensing assembly. In addition, the location of the segment 211 may be offset as described above. Alternatively, the segment 211 may be placed in the center of the catheter member 202. Given this configuration, the lumen for the dilation member (not shown) is offset.

The degree to which the segment 211 and sensing tip 208 extend from the catheter member 202 may vary depending on the particular application. For example, in certain variations, the sensing tip maybe immediately distal to the end of the catheter member. In alternate variations, the sensing tip may extend as shown in the drawings. Such a construction is useful when the practitioner desires to insert the sensing tip 208 into an opening within the tissue to perform additional scanning.

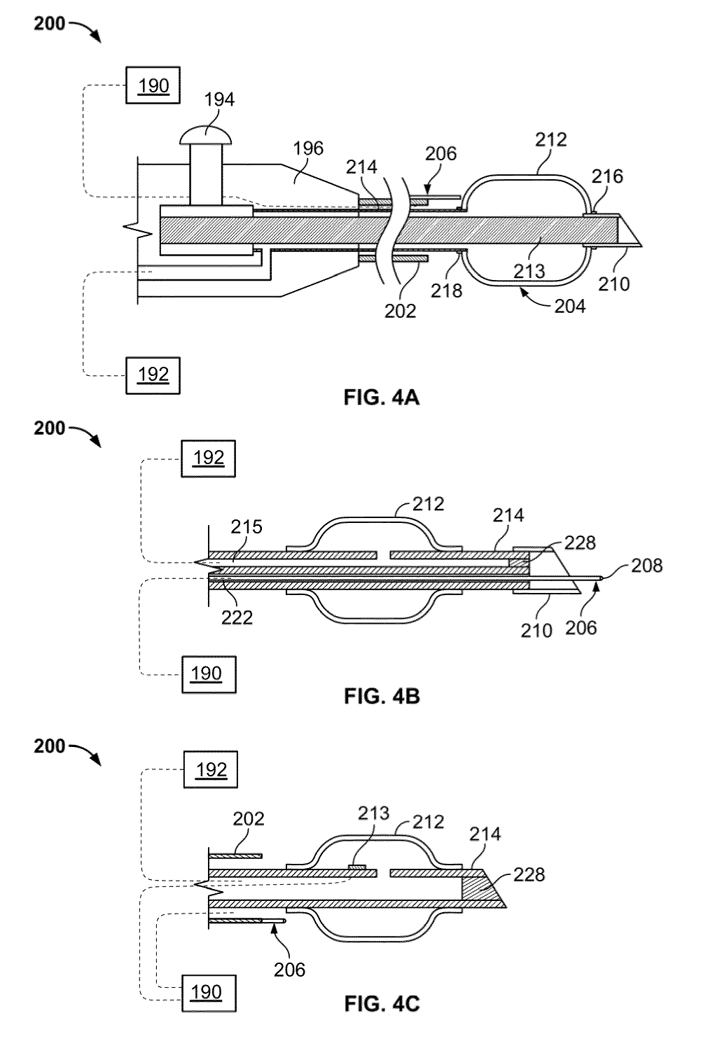

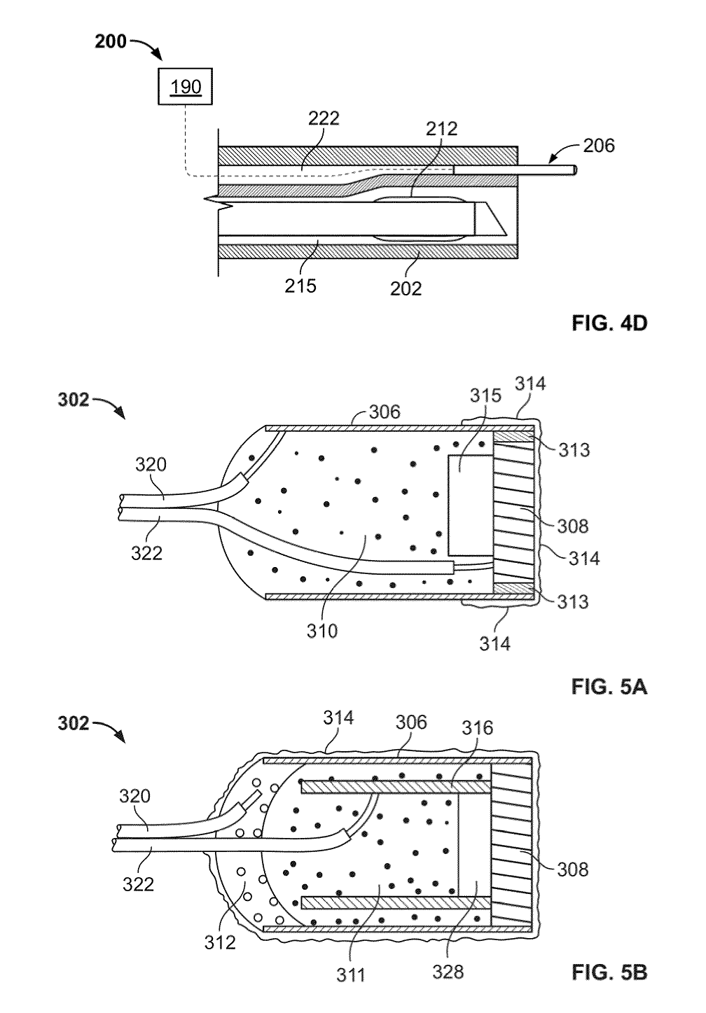

FIGS. 4A-4C illustrate various exemplary constructions of the device 200. In all cases, for illustrative purposes, the expandable member 212 is shown as being in an expanded state. During insertion of the device into the body, the expandable member 211 can be reduced to fit within the catheter member 212 or to the same size as the catheter member.

FIG. 4A shows a cross sectional view of a variation of a device 200 as described herein where the dilation member 204 is actuated. In this variation, the sensing assembly 206 is affixed to an exterior of the catheter member 202 and coupled to the control unit 190 via wires or other conducting members. As shown, the dilation member 204 is affixed to an actuator 194. However, the dilation member 204 is reinforced with a support member 213 that extends from the distal tip 210 through to the handle portion 196. To actuate the dilation member 204, the operator advances the actuator 194 to push a polymeric shaft 214 and the support member 213. As a result, the support member 213 carries the load and increases the amount of force transferred to the distal tip 210. In an additional variation, a distal end of the support member 213 may be used without a distal tip 210. In such a case, the distal end of the support member 213 may be sharpened or otherwise configured to be equivalent to the distal tip 210 of the dilation member 204. This variation also shows the distal end of the expandable member 212 being coupled to the distal tip 210 and the proximal end being coupled to the shaft 214. While both ends of the balloon may be constricted to be the same size, the distal end of the balloon may be a smaller size to ease insertion of the tip 210 and distal end of the un-inflated balloon into the tissue.

The support member may be a flexible mandrel or tube. Alternatively, it may be a braided member that provides flexibility for navigation through tortuous anatomy and column strength for driving the end of the device into tissue.

The use of a reinforcing or support member 213 provides the device 200 with considerable flexibility to navigate through tortuous anatomy while maintaining greater column strength over a device having a non-reinforced polymer shaft. However, in certain variations, a reinforcing member may be incorporated into the shaft 214. In alternative variations, no reinforcing member is used.

FIG. 4A, also illustrates the interior of the expandable member being coupled to the fluid source 192. The illustration is intended for exemplary purposes as any number of couplings may be employed to fluidly couple the expandable member to the fluid source.

FIG. 4B illustrates another variation of a shaft 214 of a dilation member. In this example the catheter member is omitted. As shown, a first lumen 215 fluidly couples the fluid source 192 to the expandable member 212. A second lumen 222 carries the sensing assembly 206. In this variation, the sensing assembly 206 is affixed to a catheter member so that the dilation member moves over the sensing assembly 206 to penetrate tissue. As also shown, the first lumen 215 may be sealed with a plug 228 or other occlusion member to prevent fluid from escaping from the device.

FIG. 4C shows another variation of a device 200 in which the shaft 214 has a single lumen 215 that couples the fluid source 192 to the expandable member 212. As with the previous variation, the lumen 215 may be plugged or occluded 228. The sensing assembly 206 of this variation is located on the catheter member 202. As noted herein, the coupling between the sensing assembly 206 and the control system 190 may go through the catheter member 202 or through a wall of the catheter member 202.

FIG. 4C also illustrates another aspect in which the device 200 includes a sensor or transducer (as described herein) 213 within the expandable member 212. As described below, placement of a sensor or transducer 213 within the expandable member 212 permits the ability to perform additional scanning of the tissue prior to placement of any implants.

FIG. 4D shows a variation of a device 200 in which two lumens 215 and 222 extend through the catheter member 202. However, the second lumen 222 has a reduced cross sectional area towards the far end of the catheter 202. This configuration permits increased clearance for the expandable member 212. In use, the device 200 creates multiple openings during a single session. Increasing the size of the first lumen 215 provides more clearance for the expandable portion of the dilation member to re-enter the catheter member 202.

The devices described above may be constructed from any standard medical grade material. For example, the shafts and catheters may comprise commercially available medical-grade flexible tubing. For example, the elongate member may comprise PTFE, polyimide, polypropylene, or other such engineered polymers such as Hytrel® manufactured by DuPont.

FIGS. 5A-5B illustrate a non-exhaustive sample of variations of transducer assemblies 302 configured to reduce an overall size of the assembly. It is noted that the invention may use any type of transducer assembly. FIG. 5A illustrates a cross-sectional view of a basic variation of a transducer assembly 302. The transducer assembly 302 includes at least one transducer 308 (e.g., a piezoelectric transducer.) In this variation, the front surface of the transducer 308 comprises a first pole and the rear surface comprises a second pole.

The transducer or transducers may comprise a piezo-ceramic crystal (e.g., a Motorola PZT 3203 HD ceramic). In the current invention, a single-crystal piezo (SCP) is preferred, but the invention does not exclude the use of other types of ferroelectric material such as poly-crystalline ceramic piezos, polymer piezos, or polymer composites. The substrate, typically made from piezoelectric single crystals (SCP) or ceramics such as PZT, PLZT, PMN, PMN-PT, also, the crystal may be a multi layer composite of a ceramic piezoelectric material. Piezoelectric polymers such as PVDF may also be used. Micromachined transducers, such as those constructed on the surface of a silicon wafer are also contemplated (e.g., such as those provided by Sensant of San Leandro, CA.) As described herein, the transducer or transducers used may be ceramic pieces coated with a conductive coating, such as gold. Other conductive coatings include sputtered metal, metals, or alloys, such as a member of the Platinum Group of the Periodic Table (Ru, Rh, Pd, Re, Os, Ir, and Pt) or gold. Titanium (Ti) is also especially suitable. The transducer may be further coated with a biocompatible layer such as Parylene or Parylene C.

The covering 306 of the transducer assembly 302 may contain at least a portion of the transducer 308. In some variations of the invention, the covering 306 may comprise a conductive material. In such cases the covering 306 itself becomes part of the electrical path to the first pole of the transducer 308. Use of a conductive covering 306 may require insulating material 313 between the sides of the transducer 308, thereby permitting a first conductive medium 314 to electrically couple only one pole of the transducer 308 to the covering 306.

At least a portion of the front surface of the transducer 308 will be in contact with the conductive medium 314. The conductive medium 314 permits one of the poles of the transducer 308 to be placed in communication with a conducting member that is ultimately coupled to a power supply. As shown in this example, the conductive medium 314 places the pole of the transducer 308 in electrical communication with the covering 306. In some variations the conductive medium 314 may coat the entire transducer 308 and covering 306. Alternatively, the conductive medium 314 may be placed over an area small enough to allow for an electrical path between a conducting member and the respective pole of the transducer 308. The conductive medium 314 may be any conductive material (e.g., gold, silver, tantalum, copper, chrome, or any bio-compatible conductive material, etc. The material may be coated, deposited, plated, painted, wound, wrapped (e.g., a conductive foil), etc. onto the transducer assembly 302.

The transducer assembly 302 depicted in FIG. 5A also illustrates conducting members 320, 322 electrically coupled to respective poles of the transducer 308. Optionally, the conducting members 320, 322 may be encapsulated within an epoxy 311 located within the covering 306. The epoxy 311 may extend to the transducer 308 thereby assisting in retaining both the conducting members 320, 322 and transducer 308 within the covering. It may also be desirable to maintain a gap 328 between the transducer 308 and any other structure. It is believed that this gap 228 improves the ability of the transducer assembly 302 to generate a signal.

FIG. 5B illustrates another variation of a transducer assembly 302. In this variation, the conductive medium 314 extends over the entire transducer covering 306. Accordingly, the covering 306 may be made of a non-conducting material (e.g. a polyamide tube, polyetherimide, polycarbonate, etc.) The transducer assembly 302 may further comprise a second tube 316 within the covering 306. This second tube 316 may be a hypo-tube and may optionally be used to electrically couple one of the conducting members to a pole of the transducer 308. As shown, the covering 306 may contain a non-conductive epoxy 310 (e.g., Hysol 2039/3561 with Scotchlite glass microspheres B23/500) which secures both the conducting member and the second tube 316 within the covering 306. This construction may have the further effect of structurally securing the transducer 308 within the assembly 302. Again, a gap 328 may or may not be adjacent to the transducer to permit displacement of the transducer 308.

FIG. 5B also illustrates the assembly 302 as having a conductive epoxy 312 which encapsulates the alternate conducting member 320. An example of a conductive epoxy is Bisphenol epoxy resin with silver particulates to enable conductivity. The particulates may be from 70-90% of the resin composition. The resin may then be combined with a hardener (e.g., 100 parts resin per 6 parts hardener.) The conductive epoxy 312 is in electrical communication with the conductive medium 314 allowing for a conductive path from the conducting member 320 to the conductive medium 314. Accordingly, use of the conductive epoxy 312 secures the conducting member 320 to the assembly 302 while electrically coupling the conducting member 320 to the transducer via the conductive coating 314.

Although variations of the transducer assembly include a tip and housing, the invention may omit the transducer covering and other structures not necessary to generate a source signal and receive a reflected signal. Therefore, it is contemplated that the invention may simply have a transducer that is coupled to a controller.

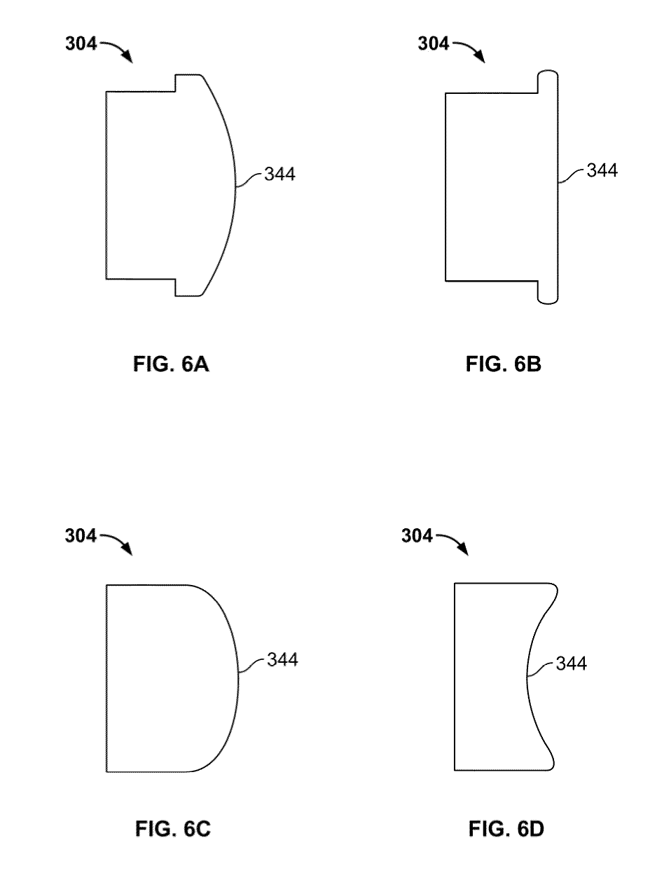

When used in the devices 200 described herein, the tip 208 of the sensing assembly may comprise the transducer 308 shown above, or the coating 314. In alternative variations, the tip 208 of the sensing assembly may comprise a tip 304 that is affixed to the transducer assembly 302 and as shown in FIGS. 6A-6D.

FIGS. 6A-6D, illustrate possible variations of tips 304 for use with the transducer assembly. It is noted that these variations are provided for illustrative purposes and are not meant to be exhaustive. The tips 304 of the present invention may function simply as a blunting tip (but still passes and receives ultrasound signals) or as a lens to disperse and/or direct the signal over a substantial portion of the outer surface of the tip 304. When configured to function as a lens, the tip 304 is adapted to disperse and/or direct (e.g., by diffraction) a reflected signal towards the transducer (not shown in FIGS. 6A-6D). Accordingly, given the above described configuration, the inventive device 300 will be able to detect vessels with substantially most of the tip 304. The tip may comprise a signal directing means.

When configured to function as a lens, the tip 304 is designed such that it interferes and redirects the signals in a desired direction in a manner like a lens. It also may be desirable to place an epoxy between the tip 304 and the transducer. Preferably, the epoxy is thin and applied without air gaps, bubbles or pockets. Also, the density/hardness of the epoxy should provide for transmission of the signal while minimizing any effect or change to the source signal. The configuration of the transducer assembly 302 permits the lens tip 304 to disperse a signal over a substantial portion of its outer surface 244. The lens tip 304 also is adapted to refract a reflected signal towards the transducer 308. Accordingly, given the above described configuration, the inventive device will be able to detect vessels with any part or substantially the entire lens tip 304 that contacts tissue.

Although the tip of the present invention is able to transmit a source signal and receive a reflected signal, the invention is not limited to requiring both functions. For example, the inventive device could be configured to generate a source signal and direct the source signal to an area of interest but a second device or transducer assembly could be used to receive the reflected signal. Accordingly, a separate device could be used to generate the source signal with the inventive device being used to receive the reflected signal.

The tip 304 may be comprised of materials such as a dimethyl pentene, a methylpentene copolymer (plastic-TPX), aluminum, carbon aerogel, polycarbonate (e.g., Lexan), polystyrene, or etc., any standard material used for ultrasound applications.

As illustrated in FIG. 6A, although the front surface 244 of the tip 302 is illustrated as being hemispherical, the tip 304 may have other profiles as well. For example, it is desirable that the tip 304 produce a certain amount of divergence of the signal being passed therethrough. However, depending on a variety of factors (e.g., material, frequency of the signal, etc.) a tip 304 may encounter excessive divergence which is destructive to the outgoing signal. Accordingly, it may be desirable to produce a tip 304 as illustrated in FIG. 6B in which a front surface 344 of the tip 304 is substantially flat. The degree of flatness of the tip 304 will often depend upon experimentation to reduce the amount of destructive reflections, thus minimizing excessive divergence due to differences in speed of sound in tip versus tissue. For example, when using a tip that is conducive to an ultrasound signal (e.g., TPX) a rounded tip can be used since there is not excessive divergence of the source signal. Use of a material that is not as conducive to ultrasound requires a flatter tip due to the resulting divergence of the source signal. FIG. 6C illustrates another variation of a tip 304 having a rounded front surface 344 but with no projections on the sides of the tip 304. FIG. 6D illustrates a tip 304 with a concave front surface 344.

In any case, the tip will be configured to avoid sharp edges that may cause any unintended damage to tissue while the device is being used to determine the presence or absence of a blood vessel. In such a case, for example, the tip may be designed such that it doesn't have sharp edges, or any sharp edges may be covered by other parts of the device (e.g., the elongate member, an outer sheath, etc.)

Commonly assigned patent publication nos. US20020128647A1; US20020138074A1; US20030130657A1, and US20050107783A1; disclose additional variations of transducer assemblies and modes of securing such assemblies to the device. The entirety of each of which is incorporated by reference herein.

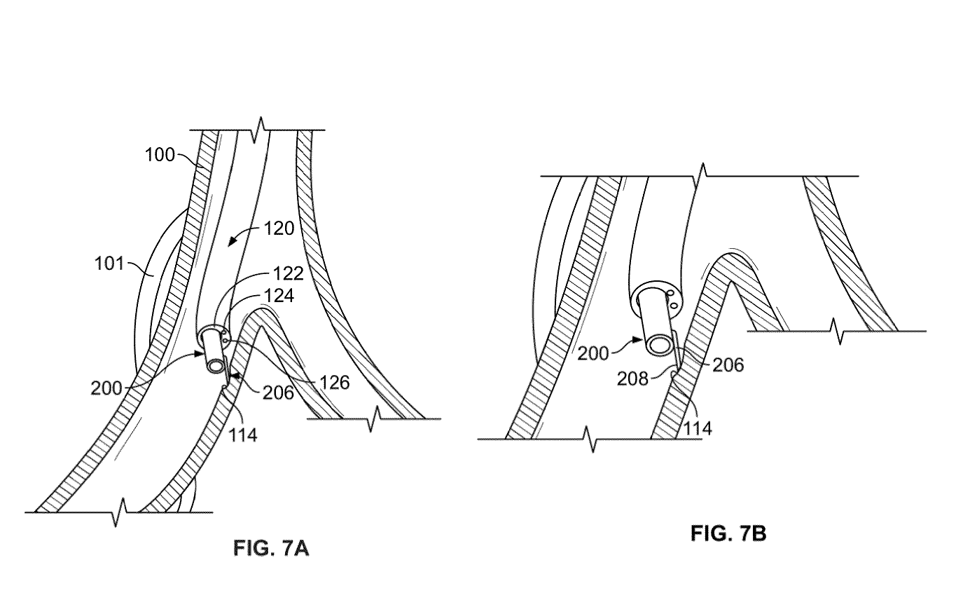

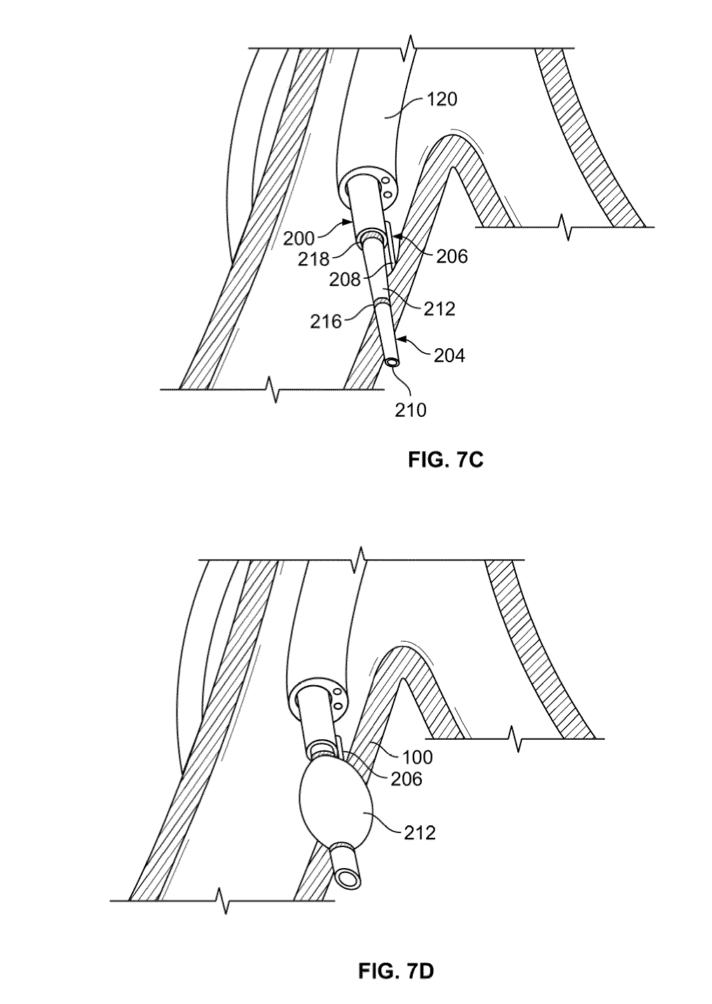

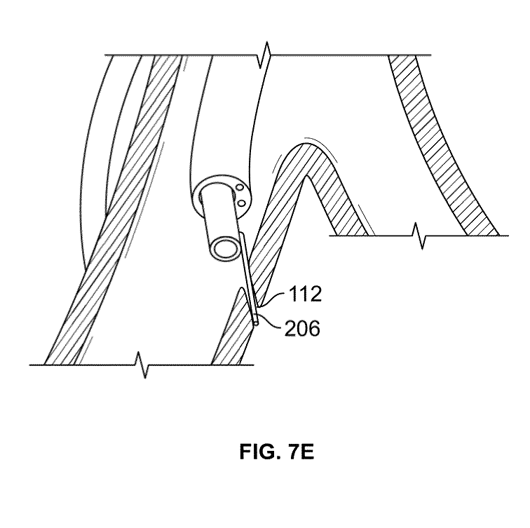

FIGS. 7A-7E illustrates an example of use of the devices described herein. Although the figures show a single variation, it is contemplated that any variation of the device may be substituted. In the illustrated example, the device creates an extra-anatomic passage in the airway wall tissue within a lung. However, it is understood that the device may be used in any part of the body and for any application. For example, variations of the device may be used during a biopsy procedure to scan for blood vessels, and remove a biopsy sample within the tissue piercing member.

FIG. 7A illustrates an access device 120 advanced into the airways 100 of a lung. The access device 120 may be a bronchoscope, endoscope, endotracheal tube with or without vision capability, or any type of delivery device. The access device 120 will have at least one lumen or working channel 122. In the illustrated version, access device 120 includes a light 124 and vision 126 capabilities. In one example, the practitioner uses the access device 120 to locate an approximate site 114 for creation of a collateral channel. For example, location of the site may be accomplished visually, or with additional equipment such as a CT scan to locate areas for treatment. In cases where the access device 120 is a bronchoscope or similar device, the access device 120 is equipped so that the surgeon may observe the site for creation of the collateral channel. In some cases it may be desirable for non-invasive imaging of the procedure. In such cases, the access device 120 as well as the other devices discussed herein, may be configured for detection by the particular non-invasive imaging technique such as fluoroscopy, “real-time” computed tomography scanning, or other technique being used.

FIG. 7A also illustrates advancement of a variation of the inventive device 200 through the channel 122 of the access device 120 towards the target site 114. The medical practitioner then uses the sensing assembly 206 to inspect the target site 114 to determine whether a blood vessel 101 is adjacent to the site. If a blood vessel is detected at or near the site 114, then another target site may be selected.

FIG. 7B shows a magnified view of the sensing assembly 206 of the device 200 being pressed against tissue at the target site 114. Accordingly, variations of the sensing assembly 206 require sufficient stiffness so that the tissue may be adequately probed. As described above, the system 150 provides the medical practitioner with audio or visual signals so that the practitioner can determine whether it is sufficiently safe to make an opening in the tissue.

FIG. 7C illustrates the device 200 after the medical practitioner adequately determines that no blood vessel 101 is adjacent to the site 114. As show, the practioner advances the dilation member 204 into the tissue. In alternate variations of the device, the sensing assembly may be used to create an opening in tissue. However, separating the tissue piercing Function from the sensing function may offer an added safety feature by preventing inadvertent puncturing of tissue. The illustration also shows another useful feature of the device 200 as the sensing assembly 204 and sensing tip 208 can remain in contact with the tissue as the distal tip 208 of the dilation member 204 advances through tissue. As noted above, this feature is useful in environments where the target tissue moves.

FIG. 7C also illustrates the use of distal and proximal markers 216, 218 on the device 200. Although the markers 216, 218 are shown to be adjacent to the expandable member 212, the markers 216, 218 may be placed anywhere along the device. However, placement of the markers 216, 218 adjacent to the expandable member 212 permits visualization of the markers 216, 218 so that the medical practitioner can minimize the chance that the proximal end of the expandable member 212 expands within the catheter member 202 or access device 120.

FIG. 7D illustrates expansion of the expandable member 212. As noted herein, the expandable member 212 may be a non-distensible balloon when the device is used in the lungs. However, variations of the device include the use of non-distensible balloons as well. FIG. 7D also illustrates deformation of the airway 100 wall upon expansion of the member 212. Accordingly, the sensing assembly 206 may or may not be displaced during this process. Furthermore, as noted above, additional variations of the device may include sensors or transducers within the balloon. In such cases, the balloon's fluid couples the internal sensor/transducer to the surrounding tissue to perform an additional scan prior to placing any implant within the dilated opening.

FIG. 7E illustrates an optional step that can take place after the dilation member 204 creates an opening 112 but before or after dilation of the opening 112. In this variation, the medical practitioner inserts the tip 208 of the sensing assembly 206 into the opening 112 to scan the area within or behind the airway wall. Accordingly, this feature is available when the size of the sensing tip 208 is less than a size of the tissue piercing member.

After dilation of the passage, the device may be removed. Alternatively, the expanded passage may be filled with fluid for additional scanning via the transducer assembly.

In one example of the procedure for placing implants in the airways to decompress hyper-inflated lungs, the medical practitioner selects a target location (usually via a CT scan) then confirms that the sensing assembly is function properly. This confirmation may be performed by placing the probe against a known region of tissue having a blood vessel. Next, the practitioner may scans the target site without knowing where a blood vessel is located. If the practitioner identifies a target site that appears to be free of blood vessels, the practioner may scan the areas around the target site to determine a vessel free region. Then, after selecting a site in the center of the region, the practioner creates the opening with a dilation member and subsequently dilates the opening. Some practitioners may scan in the opening prior to dilation and/or after dilation. Once the practitioner is satisfied that area is free of a blood vessel, the practitioner then inserts the implant.

A further variation of the invention may include configuring the transducer assembly and/or controller to have different levels of sensitivity. For example, a first level of sensitivity may be used to scan the surface of tissue. Then, after creation of the opening, the second level of sensitivity may be triggered. Such a feature acknowledges that scanning of tissue on, for example, the airway wall may require a different sensitivity than when scanning tissue within the parenchyma of the lung.

It should be noted that the invention includes kits containing the inventive device with any one or more of the following components, a Doppler ultrasound controller, a conduit as described in one or more of the applications listed above, and a bronchoscope/endoscope.

In the above explanation of Figs., similar numerals may represent similar features for the different variations of the invention.

The invention herein is described by examples and a desired way of practicing the invention is described. However, the invention as claimed herein is not limited to that specific description in any manner. Equivalence to the description as hereinafter claimed is considered to be within the scope of protection of this patent.

The devices of the present invention are configured to locate a target site for creation of a collateral channel in the tissue and to create an opening in tissue. As discussed above, a benefit of this combination feature is that a single device is able to select a target location and then create an opening without having been moved. Although the device is discussed as being primarily used in the lungs, the device is not limited as such and it is contemplated that the invention has utility in other areas as well, specifically in applications in which blood vessels or other structures must be avoided while cutting or removing tissue (one such example is tumor removal).

The above illustrations are examples of the invention described herein. It is contemplated that combinations of aspects of specific embodiments/variations or combinations of the specific embodiments/variations themselves are within the scope of this disclosure.

1. A medical device for sensing blood vessels in tissue and creating passages in the tissue, the device comprising:
a catheter member having a near end, a far end, and a lumen extending therethrough;
a dilation member within the lumen and having a shaft, the shaft having a distal tip and a non-distensible expandable member adjacent to the distal tip, the dilation member being slidably located within the catheter member;
a sensing assembly located at the far end of the catheter member; and
where the non-distensible expandable member is moveably located within the lumen independent of the sensing assembly, such that when the sensing assembly contacts tissue, the dilation member may be advanced out of the catheter member and into the tissue without removing the sensing member from the tissue.

2.-3. (canceled)

4. The medical device of claim 1, where the sensing assembly comprises a Doppler ultrasound transducer assembly.

5. The medical device of claim 1, where the sensing assembly extends through the shaft of the dilation member, such that the dilation member may advance out of the catheter member over the sensing member and into tissue without removing the sensing member from the tissue.

6. The medical device of claim 5, where an end of the sensing assembly is adapted to penetrate soft tissue.

7. The medical device of claim 1, where a segment of the sensing assembly extends from the far end of the catheter member.

8. (canceled)

9. The medical device of claim 7, where a diameter of the segment is less than a diameter of the distal tip, such that the segment may advance into an opening in tissue created by the distal tip.

10. The medical device of claim 7, where the segment has sufficient stiffness to provide force against tissue when pressed thereon.

11. The medical device of claim 10, where a portion of the sensing assembly proximal to the segment has a low column strength.

12. The medical device of claim 7, where the segment is affixed to an interior surface of the catheter member.

13.-14. (canceled)

15. The medical device of claim 1, where the distal tip of the dilation member comprises a cannula, and where the non-distensible expandable member is located about the cannula.

16. (canceled)

17. The medical device of claim 1, further comprising a tissue piercing member on the distal tip of the shaft, where the tissue piercing member is able to penetrate soft tissue.

18.-21. (canceled)

22. The medical device of claim 1, further comprising a handle at a near end of the catheter member, the handle having a first moveable actuator coupled to the dilation member.

23. The medical device of claim 1, where the catheter member further comprises a second lumen, where the sensing assembly extends through the second lumen, where the second lumen opens distally to an opening of the first lumen.

24. The medical device of claim 1, where the dilation member further comprises a support member extending therethrough, where the support member has sufficient column strength to drive the distal tip into tissue.

25. The medical device of claim 1, where the dilation member further comprises a first marker located at a distal end of the non-distensible expandable member.

26.-27. (canceled)

28. The medical device of claim 25, where the dilation member further comprises a second marker located at a proximal end of the non-distensible expandable member.

29. The medical device of claim 28, where the second marker is a visible marker.

30. The medical device of claim 28, where the second marker is a radiopaque marker.

31. The medical device of claim 1, where the dilation member is tapered where a diameter of the distal tip is less than a diameter of the shaft.

32. (canceled)

33. A system for sensing blood vessels in tissue and creating passages in the tissue, the device comprising:
a device comprising a catheter member having a near end, a far end, and a lumen extending therethrough;
a dilation member within the lumen and having a shaft, the shaft having a distal tip and a non-distensible expandable member adjacent to the distal tip, the dilation member being slidably located within the catheter member;
a Doppler ultrasound transducer assembly located at the far end of the catheter member; and
where the non-distensible expandable member is moveably located within the lumen independent of the Doppler ultrasound transducer, such that when the Doppler ultrasound transducer contacts tissue, the dilation member may be advanced out of the catheter member and into the tissue without removing the Doppler ultrasound transducer from the tissue;
a Doppler control unit adapted to be coupled to the near end of the catheter; and
a fluid source adapted to be coupled to the dilation member.

34. A method of treating lung tissue, method comprising:
selecting an area in a lung;
advancing a device into the lung to a tissue site, where the device includes a sensing assembly affixed to a catheter and a dilation assembly located within the catheter;
scanning the tissue site with the sensing assembly for the presence or absence of blood vessels;
creating an opening with the device without removing the sensing assembly from the tissue site; and
dilating the opening with the dilation assembly.

35.-44. (canceled)
